(19) United States
(12) Patent Application Publication
(10) Pub. No.: US 2009/0292178 A1
(43) Pub. Date: Nov. 26, 2009

(54) SYSTEM AND METHOD OF MONITORING USERS DURING AN INTERACTIVE ACTIVITY

(75) Inventors: Jason ELLIS, San Diego, CA (US); Clint McClellan, Del Mar, CA (US)

Correspondence Address: QUALCOMM INCORPORATED, 5775 MOREHOUSE DR., SAN DIEGO, CA, 92121, US

(73) Assignee: QUALCOMM Incorporated, San Diego, CA (US)

(21) Appl. No.: 12/124,606

(22) Filed: May 21, 2008

(51) Int. Cl. A61B 5/00

(52) U.S. Cl. 600/301

(57) ABSTRACT

A game system is disclosed and may include an exercise device and at least one sensor that may be configured to be worn by a user of the exercise device during an activity. The system may further include a game console that may be connected to the exercise device and at least one sensor. The game console may include a processor that is operable to monitor one or more vital signs associated with a player during an activity via at least one sensor and to provide feedback to the player at least partially based on the vital signs.

### FIELD

The present invention generally relates to the game systems, and more particularly, to systems and methods for monitoring users during a workout, a game, or a combination thereof.

### DESCRIPTION OF THE RELATED ART

There is a growing concern in many countries regarding the general health of people. Many people lead very unhealthy lifestyles and do not exercise on a regular basis. Further, many people purchase gym memberships or home exercise devices and do not regularly utilize their memberships or the exercise devices. It is well understood that motivation and distraction are the two main techniques that may be used to get people to use, and continue to use, fitness equipment. Gyms include instructors and trainers. Further, a gym may provide a conducive environment for friendly peer pressure that may give someone more reasons to engage in a workout. Also, at a gym someone is more likely to find a workout partner to motivate and inspire them to workout. At home, people do not have the luxury of an instructor or a trainer to provide the much needed motivation and distraction.

Accordingly, there exists a need for a system and method for addressing these deficiencies.

### SUMMARY OF THE DISCLOSURE

A method of providing an interactive activity is disclosed and may include monitoring one or more vital signs associated with a player during an activity and at least partially based on the vital signs, providing feedback to the player.

The one or more vitals may include at least one of the following: a heart rate, a body temperature, a perspiration rate, a respiration rate, a blood glucose level, a body mass index, or any combination thereof. Further, the feedback may be provided by a virtual trainer presented via a display device. In this aspect, the vital signs may be monitored using one or more sensors worn by the player. The sensors may include at least one of the following: a heart rate sensor, a body temperature sensor, a perspiration sensor, a respiration sensor, a blood glucose sensor, a body mass index sensor, or a combination thereof.

In this aspect, the method may further include comparing the vital signs to established safe parameters associated with the player, determining whether the vital signs are within a safe zone, and modifying the activity until the vital signs return to the safe zone when the vital signs are outside of the safe zone. Further, the method may include stopping the activity when the vital signs do not return to the safe zone within a time period.

The method may also include modifying an appearance of a virtual player associated with the player at least partially based on the vital signs of the player. Moreover, the method may include comparing the vital signs to a fitness threshold associated with the player; and determining whether the vital signs are above or below the fitness threshold. When the vital signs are below the fitness threshold, the method may include offering motivation to the player until the vital signs move above the fitness threshold. The motivation may be offered to the player by a virtual trainer presented via a display device. Also, when the vital signs are above the fitness threshold, the method may include offering positive feedback to the user. The positive feedback may be offered to the player by a virtual trainer presented via a display device.

In this aspect, the method may also include monitoring the movement of the player and determining whether the player is performing the activity correctly. When the player is performing the activity correctly, the method may include offering congratulations to the player. The congratulations may be offered to the player by a virtual trainer presented via a display device. When the player is not performing the activity correctly, the method may include offering one or more tips to the player to improve performance. The tips may be offered to the player by a virtual trainer presented via a display device. Further, the movement of the player may be monitored using one or more sensors worn by the player during the activity. The sensors may include at least one of the following: an accelerometer, a gyroscope, a pressure sensor, a weight sensor, a speedometer, a tachometer, a magnetometer, a pedometer, or any combination thereof.

In this aspect, the method may further include collecting the vital signs of the player, storing the vital signs of the player, determining whether a designated recipient for the vital signs is associated with the player, creating a report with the vital signs, and transmitting the report to the designated recipient associated with the player.

Further, the method may include retrieving previous activity information, and at least partially based on the previous activity information, the method may include varying an intensity of the activity. For example, the method may include increasing the intensity of the activity or decreasing the intensity of the activity.

In another aspect, a game server is disclosed and may include means for monitoring one or more vital signs associated with a player during an activity and means for providing feedback to the player at least partially based on the vital signs.

The one or more vitals may include at least one of the following: a heart rate, a body temperature, a perspiration rate, a respiration rate, a blood glucose level, a body mass index, or any combination thereof. Moreover, the feedback may be provided by a virtual trainer presented via a display device. Also, the vital signs may be monitored using one or more sensors worn by the player. The sensors may include at least one of the following: a heart rate sensor, a body temperature sensor, a perspiration sensor, a respiration sensor, a blood glucose sensor, a body mass index sensor, or a combination thereof.

The game server may further include means for comparing the vital signs to established safe parameters associated with the player, means for determining whether the vital signs are within a safe zone, and means for modifying the activity until the vital signs return to the safe zone when the vital signs are outside of the safe zone. Also, the game server may include means for stopping the activity when the vital signs do not return to the safe zone within a time period and means for modifying an appearance of a virtual player associated with the player at least partially based on the vital signs of the player. The game server may also include means for comparing the vital signs to a fitness threshold associated with the player and means for determining whether the vital signs are above or below the fitness threshold. Further, the game server may include means for offering motivation to the player until the vital signs move above the fitness threshold when the vital signs are below the fitness threshold. The motivation may be offered to the player by a virtual trainer presented via a presented display device.

The game server may also include means for offering positive feedback to the user when the vital signs are above the fitness threshold. The positive feedback may be offered to the player by a virtual trainer presented via a display device.

In this aspect, the game server may also include means for monitoring the movement of the player and means for determining whether the player is performing the activity correctly. Additionally, the game server may include means for offering congratulations to the player when the player is performing the activity correctly. The congratulations may be offered to the player by a virtual trainer presented via a display device. The game server may also include means for offering one or more tips to the player to improve performance when the player is not performing the activity correctly. The tips may be offered to the player by a virtual trainer presented via a display device. The movement of the player may be monitored using one or more sensors worn by the player during the activity. The sensors may include at least one of the following: an accelerometer, a gyroscope, a pressure sensor, a weight sensor, a speedometer, a tachometer, a magnetometer, a pedometer, or any combination thereof.

In this aspect, the game server may also include means for collecting the vital signs of the player, means for storing the vital signs of the player, means for determining whether a designated recipient for the vital signs is associated with the player, means for creating a report with the vital signs, and means for transmitting the report to the designated recipient associated with the player. Also, the game server may include means for retrieving previous activity information and means for varying an intensity of the activity at least partially based on the previous activity information. For example, the game server may include means for increasing the intensity of the activity at least partially based on the previous activity information or means for decreasing the intensity of the activity at least partially based on the previous activity information.

In yet another aspect, a game server is disclosed and may include a processor that is operable to monitor one or more vital signs associated with a player during an activity and to provide feedback to the player at least partially based on the vital signs.

The one or more vitals may include at least one of the following: a heart rate, a body temperature, a perspiration rate, a respiration rate, a blood glucose level, a body mass index, or any combination thereof. Moreover, the feedback may be provided by a virtual trainer presented via a display device. Also, the vital signs may be monitored using one or more sensors worn by the player. The sensors may include at least one of the following: a heart rate sensor, a body temperature sensor, a perspiration sensor, a respiration sensor, a blood glucose sensor, a body mass index sensor, or a combination thereof.

The processor may also be operable to compare the vital signs to established safe parameters associated with the player, to determine whether the vital signs are within a safe zone, and to modify the activity until the vital signs return to the safe zone when the vital signs are outside of the safe zone. Also, the processor within the game server may be operable to stop the activity when the vital signs do not return to the safe zone within a time period and to modify an appearance of a virtual player associated with the player at least partially based on the vital signs of the player. The processor may also be operable to compare the vital signs to a fitness threshold associated with the player and to determine whether the vital signs are above or below the fitness threshold. Further, the processor within the game server may be operable to offer motivation to the player until the vital signs move above the fitness threshold when the vital signs are below the fitness threshold. The motivation may be offered to the player by a virtual trainer presented via a presented display device.

In this aspect, the processor may also be operable to offer positive feedback to the user when the vital signs are above the fitness threshold. The positive feedback may be offered to the player by a virtual trainer presented via a display device. The processor within the game server may also operable to monitor the movement of the player and to determine whether the player is performing the activity correctly. Additionally, the processor may be operable to offer congratulations to the player when the player is performing the activity correctly. The congratulations may be offered to the player by a virtual trainer presented via a display device. Further, the processor within the game server may be operable to offer one or more tips to the player to improve performance when the player is not performing the activity correctly. The tips may be offered to the player by a virtual trainer presented via a display device. The movement of the player may be monitored using one or more sensors worn by the player during the activity. The sensors may include at least one of the following: an accelerometer, a gyroscope, a pressure sensor, a weight sensor, a speedometer, a tachometer, a magnetometer, a pedometer, or any combination thereof.

In this aspect, the processor within the game server may be operable to collect the vital signs of the player, to store the vital signs of the player, to determine whether a designated recipient for the vital signs is associated with the player, to create a report with the vital signs, and to transmit the report to the designated recipient associated with the player. Also, the processor may be operable to retrieve previous activity information and to vary an intensity of the activity at least partially based on the previous activity information. For example, the processor may be operable to increase the intensity of the activity at least partially based on the previous activity information or to decrease the intensity of the activity at least partially based on the previous activity information.

In still another aspect, a computer program product is disclosed and may include a computer-readable medium. The computer-readable medium may include at least one instruction for monitoring one or more vital signs associated with a player during an activity and at least one instruction for providing feedback to the player at least partially based on the vital signs.

The one or more vitals may include at least one of the following: a heart rate, a body temperature, a perspiration rate, a respiration rate, a blood glucose level, a body mass index, or any combination thereof. Moreover, the feedback may be provided by a virtual trainer presented via a display device. Also, the vital signs may be monitored using one or more sensors worn by the player. The sensors may include at least one of the following: a heart rate sensor, a body temperature sensor, a perspiration sensor, a respiration sensor, a blood glucose sensor, a body mass index sensor, or a combination thereof.

The computer-readable medium may further include at least one instruction for comparing the vital signs to established safe parameters associated with the player, at least one instruction for determining whether the vital signs are within a safe zone, and at least one instruction for modifying the activity until the vital signs return to the safe zone when the vital signs are outside of the safe zone. Also, the computer-readable medium may include at least one instruction for stopping the activity when the vital signs do not return to the safe zone within a time period and at least one instruction for modifying an appearance of a virtual player associated with the player at least partially based on the vital signs of the player. The computer-readable medium may also include at least one instruction for comparing the vital signs to a fitness threshold associated with the player and at least one instruction for determining whether the vital signs are above or below the fitness threshold. Further, the computer-readable medium may include at least one instruction for offering motivation to the player until the vital signs move above the fitness threshold when the vital signs are below the fitness threshold. The motivation may be offered to the player by a virtual trainer presented via a presented display device.

The computer-readable medium may also include at least one instruction for offering positive feedback to the user when the vital signs are above the fitness threshold. The positive feedback may be offered to the player by a virtual trainer presented via a display device.

In this aspect, the computer-readable medium may also include at least one instruction for monitoring the movement of the player and at least one instruction for determining whether the player is performing the activity correctly. Additionally, the computer-readable medium may include at least one instruction for offering congratulations to the player when the player is performing the activity correctly. The congratulations may be offered to the player by a virtual trainer presented via a display device. The computer-readable medium may also include at least one instruction for offering one or more tips to the player to improve performance when the player is not performing the activity correctly. The tips may be offered to the player by a virtual trainer presented via a display device. The movement of the player may be monitored using one or more sensors worn by the player during the activity. The sensors may include at least one of the following: an accelerometer, a gyroscope, a pressure sensor, a weight sensor, a speedometer, a tachometer, a magnetometer, a pedometer, or any combination thereof.

In this aspect, the computer-readable medium may also include at least one instruction for collecting the vital signs of the player, at least one instruction for storing the vital signs of the player, at least one instruction for determining whether a designated recipient for the vital signs is associated with the player, at least one instruction for creating a report with the vital signs, and at least one instruction for transmitting the report to the designated recipient associated with the player. Also, the computer-readable medium may include at least one instruction for retrieving previous activity information and at least one instruction for varying an intensity of the activity at least partially based on the previous activity information. For example, the computer-readable medium may include at least one instruction for increasing the intensity of the activity at least partially based on the previous activity information or at least one instruction for decreasing the intensity of the activity at least partially based on the previous activity information.

In another aspect, a game system is disclosed and may include an exercise device and at least one sensor that may be configured to be worn by a user of the exercise device during an activity. The system may further include a game console that may be connected to the exercise device and the at least one sensor. The game console may include a processor that is operable to monitor one or more vital signs associated with a player during an activity via the at least one sensor and to provide feedback to the player at least partially based on the vital signs.

In this aspect, the one or more vitals may include at least one of the following: a heart rate, a body temperature, a perspiration rate, a respiration rate, a blood glucose level, a body mass index, or any combination thereof. Further, the feedback may be provided by a virtual trainer via a display device coupled to the game console. The at least one sensor may include at least one of the following: a heart rate sensor, a body temperature sensor, a perspiration sensor, a respiration sensor, a blood glucose sensor, a body mass index sensor, or a combination thereof.

In this aspect of a game system, the processor may also be operable to compare the vital signs to established safe parameters associated with the player, to determine whether the vital signs are within a safe zone, and to modify the activity until the vital signs return to the safe zone when the vital signs are outside of the safe zone. The processor may further be operable to stop the activity when the vital signs do not return to the safe zone within a time period.

Further, in this aspect, the processor may be operable to modify an appearance of a virtual player associated with the player at least partially based on the vital signs of the player. Also, the processor may be operable to compare the vital signs to a fitness threshold associated with the player, and to determine whether the vital signs are above or below the fitness threshold. The processor may also be operable to offer motivation to the player until the vital signs move above the fitness threshold when the vital signs are below the fitness threshold. The motivation may be offered to the player by a virtual trainer via a display device. The processor may also be operable to offer positive feedback to the user when the vital signs are above the fitness threshold. The positive feedback may be offered to the player by a virtual trainer via a display device.

In this aspect, the processor may be operable to monitor the movement of the player using the at least one sensor and to determine whether the player is performing the activity correctly. Also, the processor may further be operable to offer congratulations to the player when the player is performing the activity correctly. The congratulations may be offered to the player by a virtual trainer presented via a display device. Moreover, the processor may be operable to offer one or more tips to the player to improve performance when the player is not performing the activity correctly. The tips may be offered to the player by a virtual trainer via a display device. In this aspect, the sensor may include at least one of the following: an accelerometer, a gyroscope, a pressure sensor, a weight sensor, a speedometer, a tachometer, a magnetometer, a pedometer, or any combination thereof.

In this aspect of a game system, the processor may also be operable to collect the vital signs of the player, to store the vital signs of the player, to determine whether a designated recipient for the vital signs is associated with the player, to create a report with the vital signs and to transmit the report to the designated recipient associated with the player. Further, the processor may be operable to retrieve previous activity information and to vary an intensity of the activity at least partially based on the previous activity information. For example, the processor may be operable to increase the intensity of the activity at least partially based on the previous activity information or to decrease the intensity of the activity at least partially based on the previous activity information.

In this aspect of the game system, the exercise device may be selected from the group comprising: a treadmill, an elliptical trainer, an elliptical cross-trainer, a stationary bike, an upright stationary bike, a recumbent stationary bike, and a rowing machine. Further, the exercise device may be selected from the group comprising: a lateral pull down device, a low row device, a biceps curl device, a triceps extension device, a leg press device, a leg extension device, a leg curl device, a squat device, a calf-raise device, a peck deck device, a flat chest press device, an incline chest press device, a decline chest press device, a shoulder press device, a cable crossover device, and a universal device.

Further aspects will be apparent in the foregoing description and equivalents thereof.

### BRIEF DESCRIPTION OF THE DRAWINGS

In the figures, like reference numerals refer to like parts throughout the various views unless otherwise indicated.

FIG. 1 is a block diagram representing an activity system; and

FIG. 2 is a first portion of a flowchart illustrating a method of providing an interactive activity;

FIG. 3 is a second portion of a flowchart illustrating a method of providing an interactive activity; and

FIG. 4 is third portion of a flowchart illustrating a method of providing an interactive activity.

### DETAILED DESCRIPTION

The word “exemplary” is used herein to mean “serving as an example, instance, or illustration.” Any aspect described herein as “exemplary” is not necessarily to be construed as preferred or advantageous over other aspects.

In this description, the term “application” may also include files having executable content, such as: object code, scripts, byte code, markup language files, and patches. In addition, an “application” referred to herein, may also include files that are not executable in nature, such as documents that may need to be opened or other data files that need to be accessed.

The term “content” may also include files having executable content, such as: object code, scripts, byte code, markup language files, and patches. In addition, an “content” referred to herein, may also include files that are not executable in nature, such as documents that may need to be opened or other data files that need to be accessed.

In this description, the terms “communication device,” “wireless device,” “wireless telephone,” “wireless communications device,” and “wireless handset” are used interchangeably. With the advent of third generation (“3G”) wireless technology, more bandwidth availability has enabled more electronic devices with wireless capabilities. Therefore, a wireless device could be a cellular telephone, a pager, a PDA, a smartphone, a navigation device, or a computer with a wireless connection. The wireless connection may be a short range wireless connection, a mid-range wireless connection, a long range wireless connection, or a combination thereof. For example, the wireless connection may be a Bluetooth connection, a Wi-Fi connection, an impulse radio connection, a cellular connection, or a combination thereof.

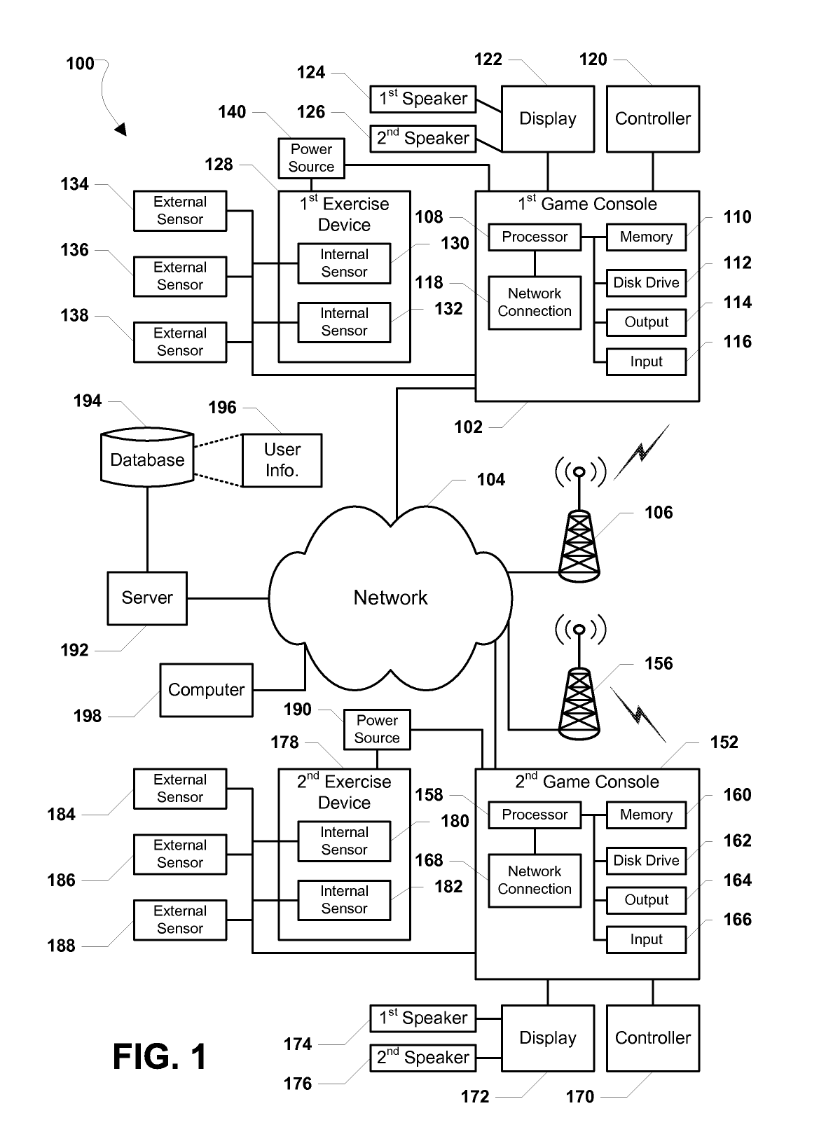

Referring initially to FIG. 1, an activity system is shown and is generally designated 100. In particular aspect, the activity system may be used to monitor one or more users, or players, during an activity, e.g., a workout, a game, some other activity, or a combination thereof. As illustrated the system 100 may include a first game console 102 connected to a network 104 via the associated communication channels. The network 104 may be any type of network. For example, the network 104 may be a wired network, a wireless network, a private network, a public network, or any combination thereof. Moreover, the associated communication channels may be any type of wired communication channel, wireless communication channel, or a combination thereof.

In a particular aspect, the first game console 102 may connect to the network 104 via a network device such as a cable modem, a dial-up modem, a digital subscriber line (“DSL”) modem, or some other network device well know in the art. In another aspect, the first game console 102 may connect to the network 104 via an over-the-air interface, e.g., a cellular tower 106, that is connected to a base station (not shown). In such a case, the first game console 102 may connect to the cellular tower via a cellular network card.

In a particular aspect, the first game console 102 may include a cradle (not shown) in which a user's wireless device (not shown), e.g., a cellular telephone, a portable digital assistant, etc., may be installed or otherwise engaged. The user's wireless device may be used to access the network 104. Further, engaging the user's wireless device may activate the system 100, as described herein, and the user's wireless device may serve as an identifier.

FIG. 1 shows that the first game console 102 may include a processor 108 therein. Further, a memory 110, i.e., a computer-readable medium, may be connected to the processor 108. By way of example, and not limitation, the memory may be a random access memory (“RAM”), a read-only memory (“ROM”), an electrically erasable programmable read only memory (“EEPROM”), a compact disc-read only memory (“CD-ROM”) or other optical disc storage, magnetic disc storage or other magnetic storage devices, or any other medium that may be used to carry or store desired program code in the form of instructions or data structures and that may be accessed by a computer. The processor 108 and the memory 110 may serve as a means for executing one or more of the method steps described herein and as a means for storing activity information, either locally or remotely. In such a case, the method steps may be stored as instructions within the memory 110 and these instructions may be executed by the processor 108.

As illustrated in FIG. 1, a disc drive 112 may also be connected to the processor 108. The disc drive 112 may be a compact disc (“CD’) drive, a digital video disc (“DVD”) drive, a DVD-ROM drive, a DVD-RAM drive, a DVD-read/write (“DVD-RW”) disc drive, or any other DVD drive well known in the art.

Further, an input 114 and an output 116 may be connected to the processor 108. The input 114 may be an input for a controller, a video input, an audio input, a sensor input, or any other type of input well know in the art. Further, the output 116 may be output for a controller, a video output, an audio output, a sensor output, or any other type of output well know in the art. The first game console 102 may also include a network device 118 installed therein. The network device 118 may be a cable modem, a dial-up modem, a digital subscriber line (“DSL”) modem, a cellular network device, or some other network device well know in the art.

As depicted in FIG. 1, the activity system 100 may further include a controller 120 connected to the first game console 108. The controller 120 may be a multi-button game controller that may allow a user to interact with the first game console 102 using his or her fingers and thumbs. The user may interact with the first game console 102 before an activity, during an activity, after an activity, or any combination thereof. FIG. 1 also shows a display 122 connected to the first game console 102. The display 122 may be a television, a computer monitor, or some other display device well known in the art. A first speaker 124 and a second speaker 126 may be connected to the display 122. In lieu of speakers 124, 126, the user may connect a pair of headphones to the display 122 or the first game console 102.

FIG. 1 also shows that the system 100 may include a first exercise device 128 connected to the first game console 102. In a particular aspect, the first exercise device 128 may be a treadmill, an elliptical trainer, an elliptical cross-trainer, a stationary bike, an upright stationary bike, a recumbent stationary bike, a rowing machine, or any combination thereof.

Further, the first exercise device 128 may be a weighted exercise device that includes a cable or belt to transfer a force to the user from a weight plate. The weighted exercise device may be a plate loaded device that does not include a cable or belt in which the load of the weight is directly transmitted to the user by one or more mechanical links. For example, the first exercise device 128 may be a lateral pull down device, a low row device, a biceps curl device, a triceps extension device, a leg press device, a leg extension device, a leg curl device, a squat device, a calf-raise device, a peck deck device, a flat chest press device, an incline chest press device, a decline chest press device, a shoulder press device, a cable crossover device, or a combination thereof (i.e., a universal device).

Additionally, the first exercise device 128 may include free weights, i.e., dumbbells, barbells, or a combination thereof. Further, the first exercise device 128 may include a racket, e.g., a tennis racket, a badminton rack, a racket ball racket, or any other racket. The first exercise device 128 may also include boxing gloves and a heavy bag, a speed bag, a double-ended bag, etc. Moreover, the first exercise device 128 may include a golf club, a baseball bat, or some other club associated with a sport. In yet another aspect, the first exercise device 128 may simply be the exercise itself in which no physical device is required, e.g., yoga, karate, etc. In such a case, the user may wear sensors that may be used as described herein to track the user's movements and the user's vital signs.

As shown, the first exercise device 128 may include a first internal sensor 130 and a second internal sensor 132. Each of the internal sensors 130, 132 may include: an accelerometer, a gyroscope, a pressure sensor, a weight sensor, a speedometer, a tachometer, a magnetometer, a pedometer, radar, a global positioning system (“GPS”) sensor, or any combination thereof. The internal sensors 130, 132 may be used to track and monitor linear movement, angular movement, linear velocity, angular velocity, linear acceleration, angular acceleration, etc. of the first exercise device 128 relative to the user, relative to the first game console 102, or a combination thereof. Further, the internal sensors 130, 132 may be used to track and monitor linear movement, angular movement, linear velocity, angular velocity, linear acceleration, angular acceleration, etc. of elements of the first exercise device 128 relative to each other, relative to the first game console 102, or a combination thereof.

The internal sensors 130, 132 may be connected to the first game console 102 via a wired connection, a wireless connection, or a combination thereof. The wired connection may include an IEEE 1394 cable (“Firewire”), a universal serial bus (“USB”) cable, an Ethernet cable, a twisted pair cable, a coaxial cable, or any other wired connection well known in the art. The wireless connection may be an 802.11/a/b/n (“Wi-Fi”) connection, a Bluetooth connection, a personal area network ultra-low-power technology (“PeANUT”) connection, an impulse radio connection, a communication link, a radar link, any combination thereof, or any other wireless connection well known in the art. Further, the wireless connection can be implemented using code division multiplexed access (“CDMA”), time division multiplexed access (“TDMA”), frequency division multiplexed access (“FDMA”), orthogonal frequency division multiplexed access (“OFDMA”), global system for mobile communications (“GSM”), Analog Advanced Mobile Phone System (“AMPS”), Universal Mobile Telecommunications System (“UMTS”), World Interoperability for Microwave Access (“WiMAX”), Bluetooth, some other wireless communication technology, or a combination thereof.

The PeANUT connection may provide a relatively low power, short range connection, i.e., ten meters (10 m) or less. Further, the PeANUT connection may provide a data rate as low as a few kilobits per second (“kbps”) per channel and as many as one megabit per second (“Mbps”) per channel with multiple channels (up to 8 Mbps). The PeANUT connection uses relatively little power, i.e., in a range of 0.005 milliWatts (“mW”) to three milliWatts (3 mW). Additionally, the PeANUT connection is relatively easily scalable and may be used to establish an ad-hoc peer-to-peer network of sensors. The PeANUT connection may operate in the globally unlicensed ultra-wideband (“UWB”) spectrum of 7.0 GHz to 8.5 GHz. Further, the PeANUT connection may transmit bits of data as series of impulses. It may be appreciated that impulse-based UWB transmission may allow for duty cycling between impulses and immunity to multipath issues. The PeANUT connection may provide strong ranging capability with accuracy of approximately one centimeter. Additionally, the PeANUT connection does not require a master-slave architecture. Any PeANUT node may send or receive data packets to or from any other PeANUT node. Further, multiple nodes may transmit and receive data simultaneously.

In a particular aspect, the internal sensors 130, 132 may track relative movement between various parts of the first exercise device 128 with respect to each other and with respect to the first game console 102 using distance ranging. In other words, by transmitting signals between the internals sensors 130, 132 and the first game console 102 and measuring the transmission time between the sensors 130, 132 or between the sensors 130, 132 and the first game console 102, the relative movement of the sensors 130, 132 may be determine with respect to each other and the first game console 102.

As shown in FIG. 1, the first exercise device 128 may include a first external sensor 134, a second external sensor 136, and a third external sensor 138. Each of the external sensors 134, 136, 138 may include: an accelerometer, a gyroscope, a pressure sensor, a weight sensor, a speedometer, a tachometer, a magnetometer, a pedometer, radar, a global positioning system (“GPS”) sensor, or any combination thereof. The external sensors 134, 136, 138 may be worn by the user, e.g., on the wrists, the ankles, around the knees, around the waist, around the head, around the chest, etc. As such, the external sensors 134, 136, 138 may be used to track and monitor linear movement, angular movement, linear velocity, angular velocity, linear acceleration, angular acceleration, etc. of the user relative to the first exercise device 102, relative to the first game console 102, or a combination thereof. Further, the external sensors 134, 136, 138 may be used to track and monitor linear movement, angular movement, linear velocity, angular velocity, linear acceleration, angular acceleration, etc. of the appendages of the user relative to the first exercise device 128, relative to each other, relative to the first game console 102, or a combination thereof. As such, the external sensors 134, 136, 138 may capture the motion of the user's appendages, e.g., arm movements, leg movements, or a combination thereof.

Each of the external sensors 134, 136, 138 may also include a heart rate sensor, a body temperature sensor, a perspiration sensor, a respiration sensor, a blood glucose sensor, a body mass index (“BMI”) sensor, a pulse rate sensor, a pulse oximetry sensor, a lung capacity sensor, or a combination thereof. Accordingly, the external sensors 134, 136, 138 may be used to sense and monitor the vital signs of the user (i.e., the “vitals”). The vitals may include a heart rate, a body temperature, a perspiration rate, a respiration rate, a blood glucose level, a BMI, a pulse rate, a hemoglobin oxygenation level, a carbon dioxide level, a lung capacity, a peak flow, or any combination thereof.

The external sensors 134, 136, 138 may be connected to the first game console 102 and the first exercise device 102 via a wired connection, a wireless connection, or a combination thereof. The wired connection may include an IEEE 1394 cable (“Firewire”), a universal serial bus (“USB”) cable, an Ethernet cable, a twisted pair cable, a coaxial cable, or any other wired connection well known in the art. The wireless connection may be an 802.11/a/b/n (“Wi-Fi”) connection, a Bluetooth connection, a personal area network ultra-low-power technology (“PeANUT”) connection, an impulse radio connection, a communication link, a radar link, any combination thereof, or any other wireless connection well known in the art. Further, the wireless connection can be implemented using code division multiplexed access (“CDMA”), time division multiplexed access (“TDMA”), frequency division multiplexed access (“FDMA”), orthogonal frequency division multiplexed access (“OFDMA”), global system for mobile communications (“GSM”), Analog Advanced Mobile Phone System (“AMPS”), Universal Mobile Telecommunications System (“UMTS”), World Interoperability for Microwave Access (“WiMAX”), Bluetooth, some other wireless communication technology, or a combination thereof.

In a particular aspect, the external sensors 134, 136, 138 may track relative movement between appendages of the user with respect to each other, with respect to the first exercise device 128, and with respect to the first game console 102 using distance ranging. In other words, by transmitting signals between the externals sensors 134, 136, 138 and the first game console 102 and measuring the transmission time between the sensors 134, 136, 138 or between the sensors 134, 136, 138 and the first game console 102, the relative movement of the sensors 134, 136, 138 may be determine with respect to each other and the first game console 102. As such, the relative movement of the user's appendages may be determined. Further, the external sensors 134, 136, 138 may be used to provide distance ranging between a first user and a second user. Moreover, the movement of a user relative to the first game console 102 may be tracked and monitored.

It may be appreciated that the first exercise device 128 may include more or less than two internal sensors 130, 132 and may include more or less than three external sensors 134, 136, 138. FIG. 1 further shows a power source 140 connected to the first exercise device 128 and the first game console 102. The power source 140 may be an alternating current (“AC”) power source, a direct current power source (“DC”), or a combination thereof. It may be appreciated that the first game console 102, the first exercise device 128, the first display 122, and the speakers 124, 126 may be included in a single device. For example, the first exercise device 128 may include the first display 122 and speakers 124, 126. Moreover, the first game console 102 may be incorporated in the first exercise device 128. Further, the system 100 may include a first game console 102 and one or more sensors connected thereto. The sensors may be connected to a user and may monitor the movement of the user, the vitals associated with the user, or a combination thereof—regardless of whether the user is utilizing the first exercise device 128.

Still referring to FIG. 1, the system 100 may also include a second game console 152 connected that is connected to the network 104. In a particular aspect, the second game console 152 may connect to the network 104 via a network device such as a cable modem, a dial-up modem, a digital subscriber line (“DSL”) modem, or some other network device well know in the art. In another aspect, the second game console 152 may connect to the network 104 via an over-the-air interface, e.g., a cellular tower 156, that is connected to a base station (not shown). In such a case, the second game console 152 may connect to the cellular tower via a cellular network card.

In a particular aspect, the second game console 152 may include a cradle (not shown) in which a user's wireless device (not shown), e.g., a cellular telephone, a portable digital assistant, etc., may be installed or otherwise engaged. The user's wireless device may be used to access the network 104. Further, engaging the user's wireless device may activate the system 100, as described herein, and the user's wireless device may serve as an identifier.

FIG. 1 shows that the second game console 152 may include a processor 158 therein. Further, a memory 160, i.e., a computer-readable medium, may be connected to the processor 158. By way of example, and not limitation, the memory may be a random access memory (“RAM”), a read-only memory (“ROM”), an electrically erasable programmable read only memory (“EEPROM”), a compact disc-read only memory (“CD-ROM”) or other optical disc storage, magnetic disc storage or other magnetic storage devices, or any other medium that may be used to carry or store desired program code in the form of instructions or data structures and that may be accessed by a computer. The processor 158 and the memory 160 may serve as a means for executing one or more of the method steps described herein and as a means for storing activity information, either locally or remotely. In such a case, the method steps may be stored as instructions within the memory 160 and these instructions may be executed by the processor 158.

As illustrated in FIG. 1, a disc drive 162 may also be connected to the processor 158. The disc drive 162 may be a compact disc (“CD”) drive, a digital video disc (“DVD”) drive, a DVD-ROM drive, a DVD-RAM drive, a DVD-read/write (“DVD-RW”) disc drive, or any other DVD drive well known in the art. Further, an input 164 and an output 166 may be connected to the processor 158. The input 164 may be an input for a controller, a video input, an audio input, a sensor input, or any other type of input well know in the art. Further, the output 166 may be output for a controller, a video output, an audio output, a sensor output, or any other type of output well know in the art. The second game console 152 may also include a network device 168 installed therein. The network device 168 may be a cable modem, a dial-up modem, a digital subscriber line (“DSL”) modem, a cellular network device, or some other network device well know in the art.

As depicted in FIG. 1, the activity system 100 may further include a controller 170 connected to the second game console 158. The controller 170 may be a multi-button game controller that may allow a user to interact with the second game console 152 using his or her fingers and thumbs. The user may interact with the second game console 152 before an activity, during an activity, after an activity, or any combination thereof. FIG. 1 also shows a display 172 connected to the second game console 152. The display 172 may be a television, a computer monitor, or some other display device well known in the art. A first speaker 174 and a second speaker 176 may be connected to the display 172. In lieu of speakers 174, 176, the user may connect a pair of headphones to the display 172 or the second game console 152.

FIG. 1 also shows that the system 100 may include a second exercise device 178 connected to the second game console 152. In a particular aspect, the second exercise device 178 may be a treadmill, an elliptical trainer, an elliptical cross-trainer, a stationary bike, an upright stationary bike, a recumbent stationary bike, a rowing machine, or any combination thereof. Further, the second exercise device 178 may be a weighted exercise device that includes a cable or belt to transfer a force to the user from a weight plate. The weighted exercise device may be a plate loaded device that does not include a cable or belt in which the load of the weight is directly transmitted to the user by one or more mechanical links. For example, the second exercise device 178 may be a lateral pull down device, a low row device, a biceps curl device, a triceps extension device, a leg press device, a leg extension device, a leg curl device, a squat device, a calf-raise device, a peck deck device, a flat chest press device, an incline chest press device, a decline chest press device, a shoulder press device, a cable crossover device, or a combination thereof (i.e., a universal device). Additionally, the second exercise device 178 may include free weights, i.e., dumbbells, barbells, or a combination thereof. Further, the second exercise device 178 may include a racket, e.g., a tennis racket, a badminton rack, a racket ball racket, or any other racket. The second exercise device 178 may also include boxing gloves and a heavy bag, a speed bag, a double-ended bag, etc. Moreover, the second exercise device 178 may include a golf club a baseball bat, or some other club associated with a sport. In yet another aspect, the second exercise device 178 may simply be the exercise itself in which no physical device is required, e.g., yoga, karate, etc. In such a case, the user may wear sensors that may be used as described herein to track the user's movements and the user's vital signs.

As shown, the second exercise device 178 may include a first internal sensor 180 and a second internal sensor 182. Each of the internal sensors 180, 182 may include: an accelerometer, a gyroscope, a pressure sensor, a weight sensor, a speedometer, a tachometer, a magnetometer, a pedometer, radar, a global positioning system (“GPS”) sensor, or any combination thereof. The internal sensors 180, 182 may be used to track and monitor linear movement, angular movement, linear velocity, angular velocity, linear acceleration, angular acceleration, etc. of the second exercise device 178 relative to the user, relative to the second game console 152, or a combination thereof. Further, the internal sensors 180, 182 may be used to track and monitor linear movement, angular movement, linear velocity, angular velocity, linear acceleration, angular acceleration, etc. of elements of the second exercise device 178 relative to each other, relative to the second game console 152, or a combination thereof.

The internal sensors 180, 182 may be connected to the second game console 152 via a wired connection, a wireless connection, or a combination thereof. The wired connection may include an IEEE 1394 cable (“Firewire”), a universal serial bus (“USB”) cable, an Ethernet cable, a twisted pair cable, a coaxial cable, or any other wired connection well known in the art. The wireless connection may be an 802.11/a/b/n (“Wi-Fi”) connection, a Bluetooth connection, a personal area network ultra-low-power technology (“PeANUT”) connection, an impulse radio connection, a communication link, a radar link, any combination thereof, or any other wireless connection well known in the art. Further, the wireless connection can be implemented using code division multiplexed access (“CDMA”), time division multiplexed access (“TDMA”), frequency division multiplexed access (“FDMA”), orthogonal frequency division multiplexed access (“OFDMA”), global system for mobile communications (“GSM”), Analog Advanced Mobile Phone System (“AMPS”), Universal Mobile Telecommunications System (“UMTS”), World Interoperability for Microwave Access (“WiMAX”), Bluetooth, some other wireless communication technology, or a combination thereof.

In a particular aspect, the internal sensors 180, 182 may track relative movement between various parts of the second exercise device 178 with respect to each other and with respect to the second game console 152 using distance ranging. In other words, by transmitting signals between the internals sensors 180, 182 and the second game console 152 and measuring the transmission time between the sensors 180, 182 or between the sensors 180, 182 and the second game console 152, the relative movement of the sensors 180, 182 may be determine with respect to each other and the second game console 152.

As shown in FIG. 1, the second exercise device 178 may include a first external sensor 184, a second external sensor 186, and a third external sensor 188. Each of the external sensors 184, 186, 188 may include: an accelerometer, a gyroscope, a pressure sensor, a weight sensor, a speedometer, a tachometer, a magnetometer, a pedometer, radar, a global positioning system (“GPS”) sensor, or any combination thereof. The external sensors 184, 186, 188 may be worn by the user, e.g., on the wrists, the ankles, around the waist, around the head, around the chest, etc. As such, the external sensors 184, 186, 188 may be used to track and monitor linear movement, angular movement, linear velocity, angular velocity, linear acceleration, angular acceleration, etc. of the user relative to the second exercise device 152, relative to the second game console 152, or a combination thereof. Further, the external sensors 184, 186, 188 may be used to track and monitor linear movement, angular movement, linear velocity, angular velocity, linear acceleration, angular acceleration, etc. of the appendages of the user relative to the second exercise device 178, relative to each other, relative to the second game console 152, or a combination thereof.

Each of the external sensors 184, 186, 188 may also include a heart rate sensor, a body temperature sensor, a perspiration sensor, a respiration sensor, a blood glucose sensor, a body mass index (“BMI”) sensor, a pulse rate sensor, a pulse oximetry sensor, a lung capacity sensor, or a combination thereof. Accordingly, the external sensors 184, 186, 188 may be used to sense and monitor the vital signs of the user (i.e., the “vitals”). The vitals may include the user's heart rate, body temperature, perspiration rate, respiration rate, blood glucose, BMI, a pulse rate, a hemoglobin oxygenation level, a carbon dioxide level, a lung capacity, a peak flow, or any combination thereof.

The external sensors 184, 186, 188 may be connected to the second game console 152 and the second exercise device 152 via a wired connection, a wireless connection, or a combination thereof. The wired connection may include an IEEE 1394 cable (“Firewire”), a universal serial bus (“USB”) cable, an Ethernet cable, a twisted pair cable, a coaxial cable, or any other wired connection well known in the art. The wireless connection may be an 802.11/a/b/n (“Wi-Fi”) connection, a Bluetooth connection, a personal area network ultra-low-power technology (“PeANUT”) connection, or any other wireless connection well known in the art. Further, the wireless connection can be implemented using code division multiplexed access (“CDMA”), time division multiplexed access (“TDMA”), frequency division multiplexed access (“FDMA”), orthogonal frequency division multiplexed access (“OFDMA”), global system for mobile communications (“GSM”), Analog Advanced Mobile Phone System (“AMPS”), Universal Mobile Telecommunications System (“UMTS”), World Interoperability for Microwave Access (“WiMAX”), Bluetooth, some other wireless communication technology, or a combination thereof.

In a particular aspect, the external sensors 184, 186, 188 may track relative movement between appendages of the user with respect to each other, with respect to the second exercise device 178, and with respect to the second game console 152 using distance ranging. In other words, by transmitting signals between the externals sensors 184, 186, 188 and the second game console 152 and measuring the transmission time between the sensors 184, 186, 188 or between the sensors 184, 186, 188 and the second game console 152, the relative movement of the sensors 184, 186, 188 may be determine with respect to each other and the second game console 152. As such, the relative movement of the user's appendages may be determined.

It may be appreciated that the second exercise device 178 may include more or less than two internal sensors 180, 182 and may include more or less than three external sensors 184, 186, 188. FIG. 1 further shows a power source 190 connected to the second exercise device 178 and the second game console 152. The power source 190 may be an alternating current (“AC”) power source, a direct current power source (“DC”), or a combination thereof. It may be appreciated that the second game console 152, the second exercise device 178, the display 172, and the speakers 174, 176 may be included in a single device. For example, the first exercise device 128 may include the first display 122 and speakers 124, 126. Moreover, the first game console 102 may be incorporated in the first exercise device 128.

It may be appreciated that with the two game consoles 102, 152 and the two exercise devices 128, 178 two users may compete with each other during an activity. It may be further appreciated that the system 100 may include more or less than two game consoles 102, 152 and two exercise devices 128, 178. As such, the system 100 may allow more than two players to compete in a single activity. Two or more players may engage in an activity using the same game console 102, 152 either directly or through a network connection, i.e., one player is remotely located, but connected to the game console 102, 152 via a network. Further, two or more players may engage in an activity using a remote game console 102, 152. These players may connect to the game console 102, 152 via a network. Additionally, two or more exercises devices 128, 178 may be connected to a single game console 102, 152, either locally, remotely, or a combination thereof.

The users may be co-located or they may be remotely located. The users may engage in competitive activities with each other. For examples, the users may compete against each other and virtual competitors in a foot race, a bike race, a rowing race, a kayaking race, etc. Further, the users may engage in a virtual boxing match, a virtual tennis match, a virtual badminton match, a virtual golf match, a virtual racket ball match, or any other two person sport involving a racket or stick. Also, the users may engage in other activities such as virtual bowling, virtual horse shoe pitching, etc.

The users may interact verbally, by text messaging, by instant messaging, through sensors, or any combination thereof. Further, the system 100 described herein may provide an augmented reality. For example, a first user may be using a stationary bike and a second user may be riding a real bike through a city. Sensors associated with the second user may transmit information back to the first user, e.g., a game console and display associated with the first user, and the information from the second user may be used to augment the experience of the first user on the stationary bike. For example, a GPS sensor on the second user may be used to track the second user through a virtual city and the first user may follow the same route as the second user through the virtual city.

One or more of the various sensors 130, 132, 134, 136, 138, 180, 182, 184, 186, 188 described herein may act as a means for monitoring the exercise devices 128, 178 used by the users. The sensors 130, 132, 134, 136, 138, 180, 182, 184, 186, 188 may monitor the movement, velocity, acceleration or any combination thereof associated with the exercise devices 128, 178 and the component parts of the exercise devices 128, 178. Further, the sensors 130, 132, 134, 136, 138, 180, 182, 184, 186, 188 may monitor forces associated with the exercise devices 128, 178, e.g., how hard a user is punching a heavy bag and therefore, how hard a user is punching a virtual opponent. Also, one or more of the sensors 130, 132, 134, 136, 138, 180, 182, 184, 186, 188 may act as a means for monitoring the vitals associated with a user. The vitals may be used to tailor, or otherwise alter, the activity program as it is in progress.

For example, if the vitals show that a user is becoming tired, the activity may automatically become easier. Alternatively, if the vitals show that a user is not becoming tired, the activity may automatically become harder. In the case of powered exercise device, e.g., a treadmill, the system 100, e.g., a gaming console 102, 152 therein, may speed the treadmill up if the vitals show the user is not becoming tired. Conversely, the system 100 may slow the treadmill down if the vitals show the user is becoming too tired. Also, in the case of a resistance exercise device, e.g., a stationary bike or weights, the system 100 may alter the resistance depending on vitals. The system 100 may increase a braking force on a stationary bike if the vitals show the user is not becoming tired or the system may decrease the braking force if the vitals show the user becoming too tired. In the case that the system 100 does not have direct control of the exercise device 128, 178, the system 100 may send an instruction to the user, e.g., via a game console 102, 152 and a display 122, 172, to alter the resistance, weight, speed, etc. of the exercise device 128, 178 accordingly. Accordingly, the system may help a user achieve a preferred pulse rate or a target pulse rate.

Further, if the vitals show that a user is in physical danger the activity may become easier until the vitals indicate the user is safe or the activity may be stopped. The vitals may be collected by the system 100 and periodically, the vitals may be transmitted, in the form of a report, to a health care provider, e.g., a doctor, nurse, a physical therapist, etc. Further, the report may be transmitted to a healthcare monitoring service, a family friend, etc. The health care provider may use the vitals to remotely track the health of the patient and if there are any signs that the patient's health is failing, the health care provider may contact the user and request that the user appears for further testing. Further, the report may be used to track the user's progress and determine trends of the user progress. With this type of trending, the system may make a user's workout get progressively harder over time or the system may make a user's workout easier if there is a substantial absence of activity between workouts.

The system 100 may further include an algorithm, e.g., stored in the first game console 102, the second game console 152, the server 192, the computer 198, or any combination thereof. The algorithm may be use to process the activity information, e.g., the user's vitals, and determine if any health issues exist for the user. If so, the algorithm may notify the appropriate individual, or individuals, regarding any issues. Also, the algorithm may be executed locally (i.e., on the game console 102, 152), remotely (i.e., on the server 192 or computer 198), or a combination thereof.

FIG. 1 further indicates that the system 100 may include a server 192 connected to the network 104. A database 194 may be connected to the server 192 and may be used to store user information 196. The user information 196 may include game information, game statistics, virtual character information, and vitals associated with one or more users. The user information 196 may be tied to an electronic medical record (“EMR”) associated with the user and stored in the database 194. The data within the database 194 may be secure, private, and the user may restrict access to the user information 196. The system 100 may also include a computer 198 connected to the network 104. The computer 198 may be used by a user to access his or her user information. Further, the computer 198 may be used by a health care professional, e.g., a doctor or a nurse, to access the user information of a particular patient. The computer 198 may also be used by a user to register with the system 100, as described below, in order to input relevant personal information and relevant fitness information, e.g., age, weight, BMI, fitness level, fitness goals, etc.

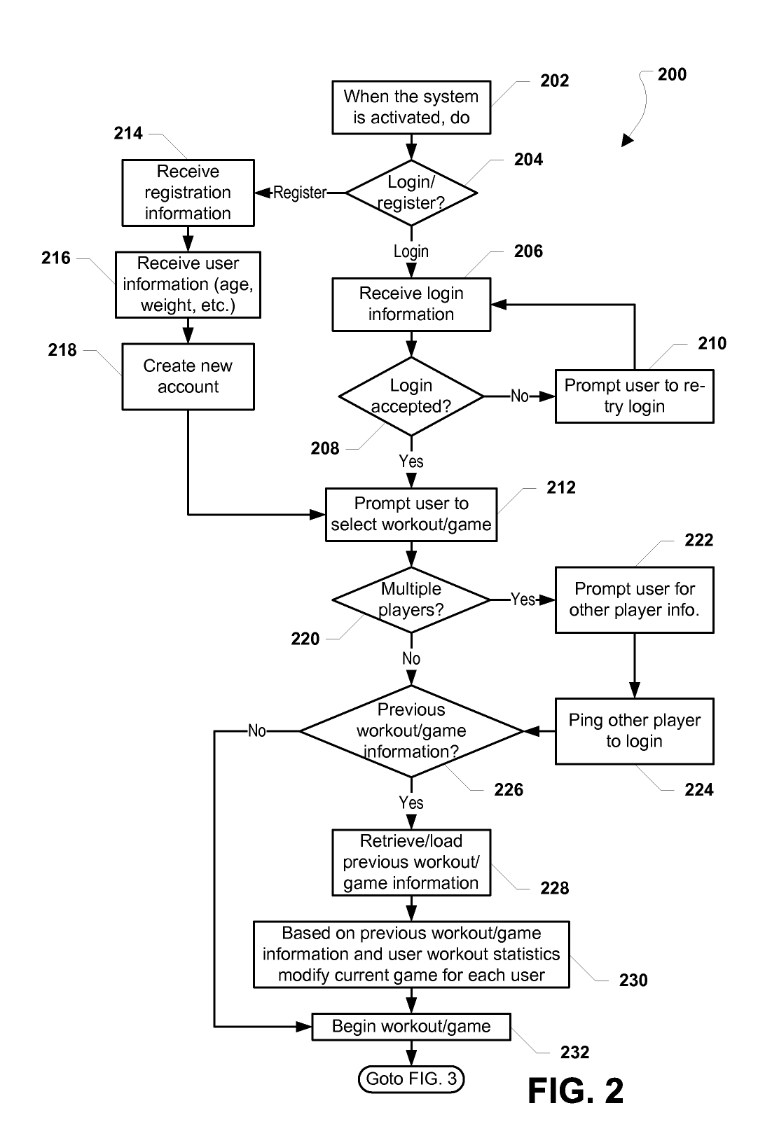

Referring now to FIG. 2 through FIG. 4, a flowchart illustrating a method of providing an interactive activity, generally designated 200. Commencing at block 202, a do loop is entered wherein when the system is activated, the following steps may be performed. In a particular aspect, the system may be activated after a user loads an activity disc in a disc drive of a game console. For example, the disc can be a Blu-Ray disc (e.g., as used by in a Sony PlayStation 3), a DVD disc (e.g., as used in a Wii), a cartridge (e.g., as used in Nintendo DS), etc.

Alternatively, the activity may be preloaded in the game console. Further, the game may be stored remotely on a game server and the game console may be used to access the game via a data network.

At decision step 204, the system may query the user to determine whether the user wishes to login or register as a new user. If the user chooses to login, the method may move to block 206 and the system may receive login information from the user. The login information may include an identifier, e.g., a username, and a password. Additionally, the login may be performed by means of a key, a proximity key, voice confirmation, fingerprint confirmation, confirmation of any other identifiable vital sign, or any combination thereof.

Moving to decision step 208, the system may determine whether the login is accepted. If not, the system may prompt the user to re-try, or re-enter, the login information. Then, the method may return to block 206 and continue as described herein. Returning to decision step 208, if the login is accepted, the method may proceed to block 212 and the system may prompt the user to select an activity.

Returning to decision step 204, if the user chooses to register, the method may move to block 214 and the system may receive registration information from the user. The registration information may include a username, a password, or a combination thereof. During registration, the system may also require that the user attach one or more sensors to his or her body, as described herein, in order to capture the resting vitals associated with the user. Accordingly, a current level of fitness for a new user may be established. By measuring the vitals periodically on an ongoing basis, the level of fitness of the user may be tracked and provided to a healthcare provider, as described herein.

At block 216, the system may receive user information from the user. The user information may include an age, a weight, a fitness level, workout goals, etc. At block 218, the system may create a new account associated with the user. The method may then move to block 212, and the system may prompt the user to select an activity. The activity may be selected by the user at the game console, the exercise device, by a remote game console, or by any combination thereof. Alternatively, the activity may be selected by a third party associated with the user, e.g., a coach, physician, physical therapist, etc.

Proceeding to decision step 220, the system may query the user to determine whether the user wishes to engage in an activity with multiple players. If so, the method may move to block 222, and the user may be prompted for the other player's, or players', information. Then, at block 224, the system may ping the other player, or players, to instruct them to login. The method may then move to block 226.

Returning to decision step 220, if the user does not wish to engage in a multi-player game, the method may move directly to decision step 226. At decision step 226, the system may determine if there is any previous activity information associated with the player, or players. If so, the method may move to block 228 and the system may retrieve the previous activity information from a database and load the previous activity information into the current game. At block 230, based on the previous activity information and user statistics, the system may modify the current game for each player, or user. For example, if the user recently performed a very intense workout, the system may modify the current workout to make it easier, or harder, depending on the fitness level of the user. Further, if there has been a substantial lapse in time between the previous workout and the current workout, the system may modify the current workout to make it relatively easier than usual. At block 232, the activity. Accordingly, the system may track a user's progress and ability and vary the intensity of an activity based on the progress and ability.

Returning to decision step 226, if the system determines that there is not any previous activity information associated with the player, or players, the method may move directly to block 232 and continue as described herein.

Moving to block 234, the system may monitor the vitals from each player. In addition to monitoring the vitals from each player, the system may display the vitals from each player. Further, the system may display the progress of each player. At block 236, at least partially based on the vitals, the system may modify the appearance of a virtual character associated with each player. For example, as the respiration of a player increases, the system may show the virtual player breathing harder. Further, as the perspiration of a player increases, the system may show the virtual player sweating more. Also, as the vitals show the player getting tired, the system may show the virtual player getting tired. Further, at least partially based on the vitals, a health status, a life status, or a combination thereof, associated with a virtual player may be altered during the activity.

Proceeding to block 238, the system may compare the vitals of each player to previously established safe parameters. The safe parameters may be determined at least partially based on the age, weight, and fitness level of each player. At decision step 240, the system may determine if the vitals of each player are outside a safe zone, a target zone, or a combination thereof. If so, the method may move to block 242 and the system may modify the activity until the vitals are within the safe zone, the target zone, or a combination thereof. Thereafter, the method may move to block 244.

Returning to decision 240, if the vitals are within the safe zone, the method may move directly to block 244. At block 244, the system may compare the vitals for each player to a predetermined threshold associated with each player. The predetermined threshold may be at least partially based on the fitness level of each player and the fitness goals associated with each player. For example, the threshold may be a target heart rate associated with a player.

At decision step 246, the system may determine whether the vitals for any player are below the associated threshold. If so, the method may move to block 248 and the system may offer motivation to the player. In a particular aspect, the system may offer motivation to the player via a virtual trainer presented to the player at a display. Thereafter, the method may move to decision step 250.

Returning to decision step 246, if the vitals of each player are not below the associated threshold, the method may move directly to decision step 250. At decision 250, the system may determine whether the vitals for any player are above the associated threshold. If so, the method may move to block 252, and the system may offer positive feedback to the player, or user, via the virtual trainer presented to the player at the display.

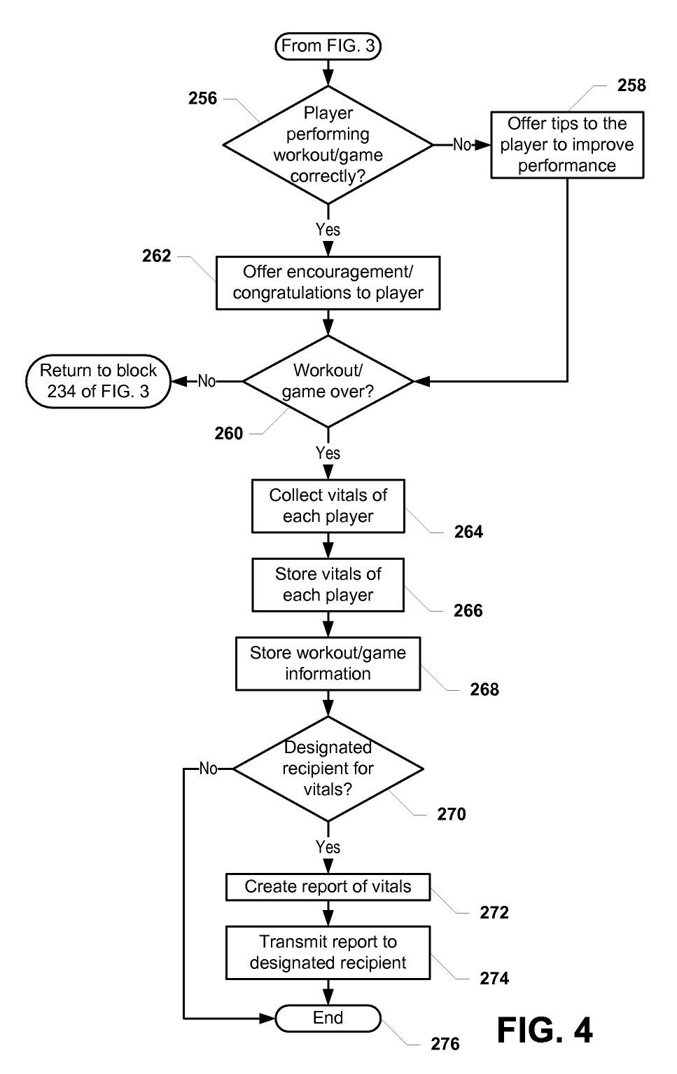

At block 254, the system may monitor the movement of each player. The system may monitor the movement based on information received from the sensors connected to each player. Thereafter, the method may proceed to FIG. 4 and continue as described herein.

Returning to decision step 250, if the vitals for any player are not above the associated threshold, the method may proceed directly to block 254 and continue as described.

Moving now to decision step 256, shown in FIG. 4, the system may determine whether any player is performing the activity correctly. The system may make this determination at least partially based on the movements of the player. If any player is not performing the activity correctly, the method may move to block 258 and the system may offer tips to the player to improve his or her performance. In a particular aspect, the tips may be offered to the player via the virtual trainer. For example, the tips may be offered via audio prompts, visual graphics, or a combination thereof. Further, the tips may be offered via text messaging, instant messaging, email, or a combination thereof. The tips may inform the user of the correct way to perform the activity. Also, the tips may include a graphical representation of the correct way to perform the activity overlaid with the actual way the user is performing the activity. From block 258, the method may move to decision step 260 and continue as described herein.

Returning to decision step 256, if each player is performing the activity properly, the method may move to block 262 and the system may offer encouragement, or congratulations, to each player. The system may offer the encouragement, or congratulations, to each player using the virtual trainer. From block 262, the method may move to decision step 260.

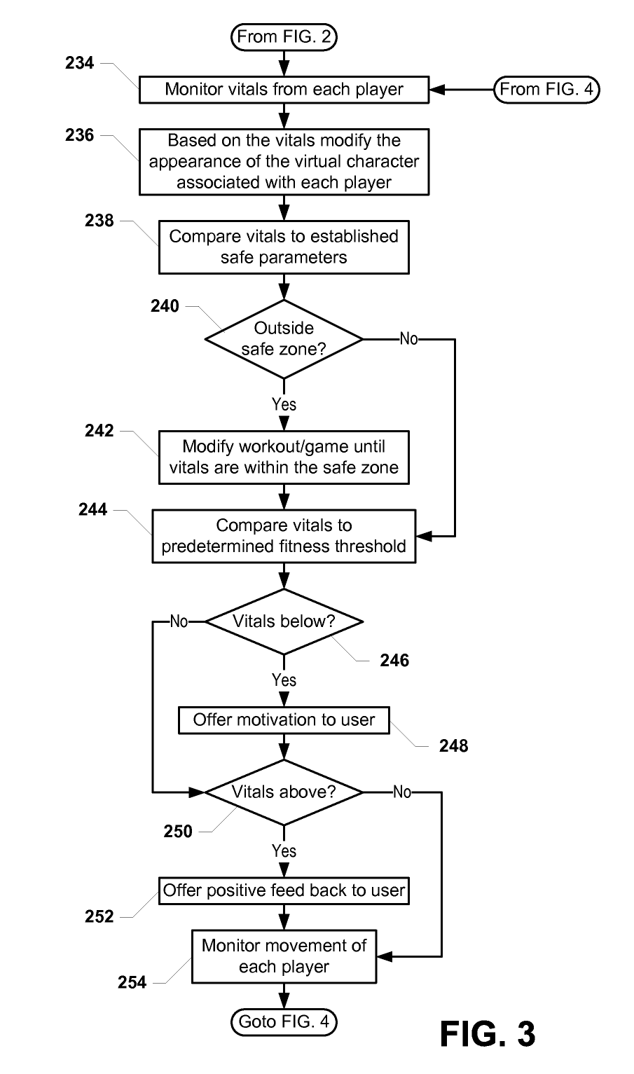

At decision step 260, the system may determine whether the activity is over. If not, the method may return to block 234 of FIG. 3 and continue as described herein. If the activity is over, the method may move to block 264 and the system may collect the vitals associated with each player. At block 266, the system may store the vitals of each player, e.g., in a database. Next, at block 268, the system may store the activity information, e.g., in the database.

It may be appreciated that a cell phone device, or other similar wireless device, may be worn by the user and may communicate with one or more sensors worn by the user during a workout, with or without an exercise device. The cell phone device may collect information relevant to the user's workout and transmit that information to a third party, e.g., a physical therapist, a doctor, a nurse, a remote trainer, etc. Accordingly, the user may perform a prescribed workout, collect information about that workout, and transmit information regarding that workout to a third party who may determine if the user is performing the workout as prescribed.

Proceeding to decision step 270, the system may determine whether any player includes a designated recipient for the vitals. The designated recipient may be a doctor, a nurse, the player, some other person, or a combination thereof. If there is a designated recipient, the method may continue to block 272 and the system may create a report of the vitals for the associated player. Thereafter, at block 274, the system may transmit the report to the designated recipient. The report may be transmitted via email, text messaging (“SMS”), instant messaging (“IM”), or any combination thereof. Also, access may be provided to the report via a web browser and may require a login. In such a case, the login information may be transmitted to the designated recipient with instructions on how to access the report. The method may end at state 276.

In a particular aspect, the report may include information regarding the exercise(s) performed, the duration of the exercise(s), vital signs, etc. Further, the report may providing trending information, i.e., whether the user's performance becomes better or worse over time. Also, the report may provide information about the user's activity as it relates to other people, i.e., persons having similar physical characteristics and fitness levels. The report may also provide information about how the user's performance relates to baseline fitness levels and vitals established, or otherwise determined, during registration.

Returning to decision step 270, if each player does not include a designated recipient to receive the respective vitals, the method may move directly to state 276 and end.

With the configuration described herein, the system and method may provide a way to monitor one or more users, or a players, during an interactive activity. Vital signs associated with the user may be monitored and the activity may be modified, or even stopped, accordingly. Further, the movement of the user may be monitored and the user may be given appropriate instruction if the movement of the user does not correspond to the correct performance of a given activity or exercise, e.g., yoga, dancing, golf, tennis, boxing, karate, etc. Additionally, the vital signs of the user may be collected during the activity and sent to the appropriate health care provider for analysis. The vital signs of the user may also be collected during rest and the resting vital signs may be used to analyze the level of fitness of the user.

The system and method described herein may be used to monitor an athlete's progress during training and to provide a verifiable record of the athlete's progress. As such, in the event an athlete becomes accused of utilizing performance enhancing substances, the information provided by the system and method described herein may be used as evidence to exonerate the user of any charges or to substantiate any charges. For example, a report provided by the system and method may display trending information showing a slow increase in performance and not any spikes in performance typically associated with the use of performance enhancing substances.

It may be understood that the method steps described herein do not necessarily have to be performed in the order as described. Further, words such as “thereafter”, “then”, “next”, etc. are not intended to limit the order of the steps. These words are simply used to guide the reader through the description of the method steps.

In one or more exemplary aspects, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that may be accessed by a computer. By way of example, and not limitation, such computer-readable media may comprise RAM, ROM, EEPROM, CD-ROM or other optical disc storage, magnetic disc storage or other magnetic storage devices, or any other medium that may be used to carry or store desired program code in the form of instructions or data structures and that may be accessed by a computer. Also, any connection is properly termed a computer-readable medium. For example, if the software is transmitted from a website, server, or other remote source using a coaxial cable, fiber optic cable, twisted pair, digital subscriber line (“DSL”), or wireless technologies such as infrared, radio, and microwave, then the coaxial cable, fiber optic cable, twisted pair, DSL, or wireless technologies such as infrared, radio, and microwave are included in the definition of medium. Disc and disc, as used herein, includes compact disc (“CD”), laser disc, optical disc, digital versatile disc (“DVD”), floppy disc and blu-ray disc where discs usually reproduce data magnetically, while discs reproduce data optically with lasers. Combinations of the above should also be included within the scope of computer-readable media.

Although selected aspects have been illustrated and described in detail, it will be understood that various substitutions and alterations may be made therein without departing from the spirit and scope of the present invention, as defined by the following claims.

1. A method of providing an interactive activity, the method comprising:
monitoring one or more vital signs associated with a player during an activity; and
at least partially based on the vital signs, providing feedback to the player.

2. The method of claim 1, wherein the one or more vitals includes at least one of the following: a heart rate, a body temperature, a perspiration rate, a respiration rate, a blood glucose level, a body mass index, or any combination thereof.

3. The method of claim 1, wherein the feedback is provided by a virtual trainer presented via a display device.

4. The method of claim 1, wherein the vital signs are monitored using one or more sensors worn by the player.

5. The method of claim 4, wherein the sensors include at least one of the following: a heart rate sensor, a body temperature sensor, a perspiration sensor, a respiration sensor, a blood glucose sensor, a body mass index sensor, or a combination thereof.

6. The method of claim 1, further comprising:
comparing the vital signs to established safe parameters associated with the player;
determining whether the vital signs are within a safe zone; and
when the vital signs are outside of the safe zone, modifying the activity until the vital signs return to the safe zone.

7. The method of claim 6, further comprising:
stopping the activity when the vital signs do not return to the safe zone within a time period.

8. The method of claim 1, further comprising:
at least partially based on the vital signs of the player, modifying an appearance of a virtual player associated with the player.

9. The method of claim 1, further comprising:
comparing the vital signs to a fitness threshold associated with the player; and
determining whether the vital signs are above or below the fitness threshold.

10. The method of claim 9, further comprising:
when the vital signs are below the fitness threshold, offering motivation to the player until the vital signs move above the fitness threshold.

11. The method of claim 10, wherein the motivation is offered to the player by a virtual trainer presented via a display device.

12. The method of claim 9, further comprising:
when the vital signs are above the fitness threshold, offering positive feedback to the user.

13. The method of claim 12, wherein the positive feedback is offered to the player by a virtual trainer presented via a display device.

14-20. (canceled)

21. The method of claim 1, further comprising:
collecting the vital signs of the player;
storing the vital signs of the player;
determining whether a designated recipient for the vital signs is associated with the player;
creating a report with the vital signs; and
transmitting the report to the designated recipient associated with the player.

22. The method of claim 1, further comprising:
retrieving previous activity information; and
at least partially based on the previous activity information varying an intensity of the activity.

23-24. (canceled)

25. A game server, comprising:
means for monitoring one or more vital signs associated with a player during an activity; and
means for providing feedback to the player at least partially based on the vital signs.

26. The game server of claim 25, wherein the one or more vitals includes at least one of the following: a heart rate, a body temperature, a perspiration rate, a respiration rate, a blood glucose level, a body mass index, or any combination thereof.

27. The game server of claim 25, wherein the feedback is provided by a virtual trainer presented via a display device.

28. The game server of claim 25, wherein the vital signs are monitored using one or more sensors worn by the player.

29. The game server of claim 28, wherein the sensors include at least one of the following: a heart rate sensor, a body temperature sensor, a perspiration sensor, a respiration sensor, a blood glucose sensor, a body mass index sensor, or a combination thereof.

30. The game server of claim 25, further comprising:
means for comparing the vital signs to established safe parameters associated with the player;
means for determining whether the vital signs are within a safe zone; and
means for modifying the activity until the vital signs return to the safe zone when the vital signs are outside of the safe zone.

31. The game server of claim 30, further comprising:
means for stopping the activity when the vital signs do not return to the safe zone within a time period.

32. The game server of claim 25, further comprising:
means for modifying an appearance of a virtual player associated with the player at least partially based on the vital signs of the player.

33. The game server of claim 25, further comprising:
means for comparing the vital signs to a fitness threshold associated with the player; and
means for determining whether the vital signs are above or below the fitness threshold.

34. The game server of claim 33, further comprising:
means for offering motivation to the player until the vital signs move above the fitness threshold when the vital signs are below the fitness threshold.

35. The game server of claim 34, wherein the motivation is offered to the player by a virtual trainer presented via a presented display device.

36. The game server of claim 33, further comprising:
means for offering positive feedback to the user when the vital signs are above the fitness threshold.

37. The game server of claim 36, wherein the positive feedback is offered to the player by a virtual trainer presented via a display device.

38-44. (canceled)

45. The game server of claim 25, further comprising:
means for collecting the vital signs of the player;
means for storing the vital signs of the player;
means for determining whether a designated recipient for the vital signs is associated with the player;
means for creating a report with the vital signs; and
means for transmitting the report to the designated recipient associated with the player.

46. The game server of claim 25, further comprising:
means for retrieving previous activity information; and
means for varying an intensity of the activity at least partially based on the previous activity information.

47-48. (canceled)

49. A game server, comprising:
a processor, wherein the processor is operable to:
monitor one or more vital signs associated with a player during an activity; and
provide feedback to the player at least partially based on the vital signs.

50. The game server of claim 49, wherein the one or more vital signs includes at least one of the following: a heart rate, a body temperature, a perspiration rate, a respiration rate, a blood glucose level, a body mass index, or any combination thereof.

51. The game server of claim 49, wherein the feedback is provided by a virtual trainer presented via a display device.

52. The game server of claim 49, wherein the vital signs are monitored using one or more sensors worn by the player.

53. The game server of claim 52, wherein the sensors include at least one of the following: a heart rate sensor, a body temperature sensor, a perspiration sensor, a respiration sensor, a blood glucose sensor, a body mass index sensor, or a combination thereof.

54. The game server of claim 49, wherein the processor is operable to:
compare the vital signs to established safe parameters associated with the player;
determine whether the vital signs are within a safe zone; and
modify the activity until the vital signs return to the safe zone when the vital signs are outside of the safe zone.

55. The game server of claim 54, wherein the processor is operable to:
stop the activity when the vital signs do not return to the safe zone within a time period.

56. The game server of claim 49, wherein the processor is operable to:
modify an appearance of a virtual player associated with the player at least partially based on the vital signs of the player.

57. The game server of claim 49, wherein the processor is operable to:
compare the vital signs to a fitness threshold associated with the player; and
determine whether the vital signs are above or below the fitness threshold.

58. The game server of claim 57, wherein the processor is operable to:
offer motivation to the player until the vital signs move above the fitness threshold when the vital signs are below the fitness threshold.

59. The game server of claim 58, wherein the motivation is offered to the player by a virtual trainer presented via a display device.

60. The game server of claim 57, wherein the processor is operable to:
offer positive feedback to the user when the vital signs are above the fitness threshold.

61. The game server of claim 60, wherein the positive feedback is offered to the player by a virtual trainer presented via a display device.

62-68. (canceled)

69. The game server of claim 49, wherein the processor is operable to:
collect the vital signs of the player;
store the vital signs of the player;
determine whether a designated recipient for the vital signs is associated with the player;
create a report with the vital signs; and
transmit the report to the designated recipient associated with the player.

70. The game server of claim 49, wherein the processor is operable to:
retrieve previous activity information; and
vary an intensity of the activity at least partially based on the previous activity information.

71-72. (canceled)

73. A computer program product, comprising:
a computer-readable medium, comprising:
at least one instruction for monitoring one or more vital signs associated with a player during an activity; and
at least one instruction for providing feedback to the player at least partially based on the vital signs.

74. The computer program product of claim 73, wherein the one or more vitals includes at least one of the following: a heart rate, a body temperature, a perspiration rate, a respiration rate, a blood glucose level, a body mass index, or any combination thereof.

75. The computer program product of claim 73, wherein the feedback is provided by a virtual trainer presented via a display device.

76. The computer program product of claim 73, wherein the vital signs are monitored using one or more sensors worn by the player.

77. The computer program product of claim 76, wherein the sensors include at least one of the following: a heart rate sensor, a body temperature sensor, a perspiration sensor, a respiration sensor, a blood glucose sensor, a body mass index sensor, or a combination thereof.

78. The computer program product of claim 73, wherein the computer-readable medium further comprises:
at least one instruction for comparing the vital signs to established safe parameters associated with the player;
at least one instruction for determining whether the vital signs are within a safe zone; and
at least one instruction for modifying the activity until the vital signs return to the safe zone when the vital signs are outside of the safe zone.

79. The computer program product of claim 78, wherein the computer-readable medium further comprises:
at least one instruction for stopping the activity when the vital signs do not return to the safe zone within a time period.

80. The computer program product of claim 73, wherein the computer-readable medium further comprises:
at least one instruction for modifying an appearance of a virtual player associated with the player at least partially based on the vital signs of the player.

81. The computer program product of claim 73, wherein the computer-readable medium further comprises:
at least one instruction for comparing the vital signs to a fitness threshold associated with the player; and
at least one instruction for determining whether the vital signs are above or below the fitness threshold.

82. The computer program product of claim 81, wherein the computer-readable medium further comprises:
at least one instruction for offering motivation to the player until the vital signs move above the fitness threshold when the vital signs are below the fitness threshold.

83. The computer program product of claim 82, wherein the motivation is offered to the player by a virtual trainer presented via a display device.

84. The computer program product of claim 81, wherein the computer-readable medium further comprises:
at least one instruction for offering positive feedback to the user when the vital signs are above the fitness threshold.

85. The computer program product of claim 84, wherein the positive feedback is offered to the player by a virtual trainer presented via a display device.

86-92. (canceled)

93. The computer program product of claim 73, wherein the computer-readable medium further comprises:
at least one instruction for collecting the vital signs of the player;
at least one instruction for storing the vital signs of the player;
at least one instruction for determining whether a designated recipient for the vital signs is associated with the player;
at least one instruction for creating a report with the vital signs; and
at least one instruction for transmitting the report to the designated recipient associated with the player.

94. The computer program product of claim 73, wherein the computer-readable medium further comprises:
at least one instruction for retrieving previous activity information; and
at least one instruction for varying an intensity of the activity at least partially based on the previous activity information.

95-96. (canceled)

97. A game system, comprising:
an exercise device;
at least one sensor configured to be worn by a user of the exercise device during an activity; and
a game console connected to the exercise device and the at least one sensor, wherein the game console comprises:
a processor, wherein the processor is operable to monitor one or more vital signs associated with a player during an activity via the at least one sensor; and
provide feedback to the player at least partially based on the vital signs.

98. The game system of claim 97, wherein the one or more vitals includes at least one of the following: a heart rate, a body temperature, a perspiration rate, a respiration rate, a blood glucose level, a body mass index, or any combination thereof.

99. The game system of claim 97, wherein the feedback is provided by a virtual trainer presented via a display device coupled to the game console.

100. The game system of claim 97, wherein the at least one sensor includes at least one of the following: a heart rate sensor, a body temperature sensor, a perspiration sensor, a respiration sensor, a blood glucose sensor, a body mass index sensor, or a combination thereof.

101. The game system of claim 97, wherein the processor is operable to:
compare the vital signs to established safe parameters associated with the player;
determine whether the vital signs are within a safe zone; and
modify the activity until the vital signs return to the safe zone when the vital signs are outside of the safe zone.

102. The game system of claim 101, wherein the processor is operable to:
stop the activity when the vital signs do not return to the safe zone within a time period.

103. The game system of claim 97, wherein the processor is operable to:
modify an appearance of a virtual player associated with the player at least partially based on the vital signs of the player.

104. The game system of claim 97, wherein the processor is operable to:
compare the vital signs to a fitness threshold associated with the player; and
determine whether the vital signs are above or below the fitness threshold.

105. The game system of claim 104, wherein the processor is operable to:
offer motivation to the player until the vital signs move above the fitness threshold when the vital signs are below the fitness threshold.

106. The game system of claim 105, wherein the motivation is offered to the player by a virtual trainer presented via a display device.

107. The game system of claim 106, wherein the processor is operable to:
offer positive feedback to the user when the vital signs are above the fitness threshold.

108. The game system of claim 107, wherein the positive feedback is offered to the player by a virtual trainer presented via a display device.

109-114. (canceled)

115. The game system of claim 97, wherein the processor is operable to:
collect the vital signs of the player;
store the vital signs of the player;
determine whether a designated recipient for the vital signs is associated with the player;
create a report with the vital signs; and
transmit the report to the designated recipient associated with the player.

116. The game system of claim 97, wherein the processor is operable to:
retrieve previous activity information; and
vary an intensity of the activity at least partially based on the previous activity information.

117-118. (canceled)

119. The game system of claim 97, wherein the exercise device is selected from the group comprising: a treadmill, an elliptical trainer, an elliptical cross-trainer, a stationary bike, an upright stationary bike, a recumbent stationary bike, and a rowing machine.

120. The game system of claim 97, wherein the exercise device is selected from the group comprising: a lateral pull down device, a low row device, a biceps curl device, a triceps extension device, a leg press device, a leg extension device, a leg curl device, a squat device, a calf-raise device, a peck deck device, a flat chest press device, an incline chest press device, a decline chest press device, a shoulder press device, a cable crossover device, and a universal device.
